(19) United States
(12) Patent Application Publication
(10) Pub. No.: US 2004/0137520 A1
(43) Pub. Date: Jul. 15, 2004

(54) Method and system for diagnosing andropause in males

(76) Inventors: Ken Adams, Toronto (CA); Doug MacFadden, Toronto (CA)

Correspondence Address: DENISE M GLASSMEYER, YOUNG & BASILE, P.C., 3001 W. BIG BEAVER RD., SUITE 624, TROY, MI, 48084-2813, US

(21) Appl. No.: 10/470,819

(22) PCT Filed: Jan. 30, 2002
(86) PCT No.: PCT/CA02/00119

(51) Int. Cl. G01N 33/53

(52) U.S. Cl. 435/7.1

(57) ABSTRACT

In accordance with the present invention there is provided a method, device, and kit for the diagnosis and assessment of andropause and/or reduced gonadal function. The method comprises conducting a biological assay on a male fluid sample and determining the presence of one or more clinical markers of andropause at a concentration indicative of andropause. The method of the present invention is simple, rapid and non-invasive. The method can be conducted privately by the patient at home with almost instantaneous results. Based on the results the patient can then seek professional medical help leading to earlier treatment and alleviation of andropausal symptoms and any associated medical conditions.

### FIELD OF THE INVENTION

The present invention relates to the diagnosis of androgen decline in males now commonly referred to as andropause or male menopause. In particular, the present invention relates to a diagnostic method, device and test kit for indicating andropausal status and/or reduced gonadal function in males.

### BACKGROUND OF THE INVENTION

Andropause, also referred to as male menopause, is defined as a progressive decline in the production of androgenic hormones which commonly occurs in ageing adult males. The age of onset of andropause is very unpredictable. Andropause can occur at any age, but it is uncommon before age 40, and the incidence increases with each decade of life. Associated with declines in androgenic hormone production are declines in male gonadal function which tend to develop very gradually over many years. The resulting clinical picture has many similarities to the menopausal process that occurs in females in which a decline in ovarian function results in a corresponding decline in the levels of estrogen.

In both andropause and female menopause the decline in biologically active levels of the sex hormones causes the clinical signs and symptoms associated with these two conditions. The declining levels of gonadal hormones are a direct result of a progressive reduction of gonadal function leading to a decrease in the production and secretion of the sex hormones. In men declining testicular function associated with andropause lowers the levels of androgens (i.e. testosterone and its precursors and metabolites).

A decline in androgen levels in males is associated with fatigue, alterations in mood and cognition (depression, mood swings, and irritability), accelerated losses of muscle mass, weight gain, increased body fat, increased risk of joint injury, osteoporosis, fractures, decreased libido, erectile dysfunction, and harmful changes in serum lipid levels. These clinical symptoms can be successfully treated or improved with successful restoration of the androgen levels to normal physiological levels. Furthermore, restoring hormone levels has also been shown to effectively reduce and prevent the pathological diseases associated with these conditions.

For men the clinical detection and treatment of andropause is complicated by:

the unpredictable age of onset (starting in the 3rd or 4th decade to 10th decade and beyond);

the symptoms often develop very slowly and insidiously over years and often over many decades;

the symptoms of andropause often mimic changes that may normally be associated with ageing;

stress and comorbidity with many other chronic diseases produce similar symptoms that cause andropause to be overlooked;

the majority of men that experience difficulties with sexual dysfunction due to andropause are too ashamed to confide in or consult with their own physicians; and

the lack of general public awareness has meant that the vast majority of men with this condition do not seek medical treatment for their symptoms, and even when medical attention is sought, the condition frequently goes undiagnosed and thus untreated.

Focus by medical practitioners has been on the treatment of the symptoms of andropause rather than the early diagnosis of the condition. Treatments for andropause to alleviate symptoms caused by androgen deficiency include the administration of androgens. For example, testosterone derivatives such as Proviron™ may be administered orally. U.S. Pat. No. 5,861,389 discloses the use of selective aromatase inhibitors for treating androgen deficiency in men. WO 99/52533 (published Oct. 21, 1999) discloses the use of ethisterone or ethisterone derivatives for treatment of androgen deficiency in males.

Presently, there exists no simple, rapid, accurate and non-invasive test for determining andropausal status. Such a test is clearly desirable in order that health care decisions can be made to reduce the morbidity that occurs when this condition goes undiagnosed and untreated. Currently, andropausal status is determined by an invasive blood test which can only be conducted by a doctor in a health care facility. Furthermore, the results of such a test make take days to obtain as performed in an analysis laboratory. In contrast, in women menopausal status is easily diagnosed by the cessation of menstruation associated with menopause. A test assembly has even been developed for detecting a clinical marker for home diagnosis of female menopause (EP 0 736 771).

Based on the above, it would be very desirable to provide a testing procedure for symptomatic men in order that more men proceed to receive appropriate and earlier treatment for this serious medical condition. Non-symptomatic men may also test for andropause. It is advantageous to have available a diagnostic method that accurately and rapidly detects a selected clinical marker for diagnosing andropause in a cost-effective, non-invasive and private manner.

### SUMMARY OF THE INVENTION

In accordance with the present invention there is provided a method for the diagnosis and assessment of andropause and/or reduced gonadal function. The method of the present invention is simple, rapid and non-invasive. The method is a no-wash, one-step method that does not require anything but the application of a suitable fluid sample to a specifically designed diagnostic device (cassette) or strip.

The method can be conducted privately by the patient at home with almost instantaneous results. Laboratory analysis is not required. Based on the results the patient can then seek professional medical help leading to earlier treatment and alleviation of andropausal symptoms and any associated medical conditions.

In accordance with an aspect of the present invention is a diagnostic method, device and kit for accurately and rapidly detecting one or more clinical markers for diagnosing andropause which allows for such diagnosis to be performed at home without requiring invasive blood collecting methods and also affords the possibility of frequently repeating the diagnostic method without any discomfort for the male patient. The device and kit are portable and can be taken and used anywhere and are suitable for private use.

Accordingly, the method, device and kit of the present invention provide a reliable result in a short time period, minutes, with a result indicative of the andropausal condition. Also, the diagnostic method of the present invention is both safe and reliable.

In accordance with another aspect of the present invention is a non-invasive method for the diagnosis and assessment of andropause, the method comprising conducting a biological assay on a male fluid sample and determining the presence of one or more clinical markers of andropause at a concentration indicative of andropause.

According to another aspect of the invention is a method to aid in the diagnosis of andropause in a male patient comprising quantifying the amount of clinical marker in a sample of a male patient wherein a certain level of clinical marker present in the sample indicates andropause of the patient.

According to yet another aspect of the invention is a method for non-invasive detection of andropause in a male subject comprising identifying the presence of one or more clinical markers of andropause in a sample of body fluids, said method comprising the steps of (a) contacting the sample with at least one antibody specific for a clinical marker of andropause; (b) incubating the sample and antibody mixture for a sufficient period of time to allow complexing of the antibody and antigen in the sample; (c) detecting any complex; and (d) correlating any complex to the presence or absence of andropause in said male subject.

In accordance with yet another aspect of the present invention is a method for detecting the presence of an andropause clinical marker as a predictive indicator of andropause in a male patient, the method comprising;

conducting a biological assay for detecting the presence of an andropause clinical marker in a biological fluid sample from said patient; and

upon detecting the andropause clinical marker in said sample classifying said sample as positive which indicates an andropausal condition.

In accordance with a further aspect of the present invention is a diagnostic method for determining the andropausal status of a male patient by detecting a clinical marker of andropause, the method comprising the steps of:

applying a biological fluid sample of said patient to a first region on a support where is located a labelled clinical marker-binding compound, wherein any clinical marker present in said biological fluid sample binds to said labelled clinical marker-binding compound forming a complex;

allowing the biological fluid sample to flow to a second region adjacent said first region where is located an antibody that recognizes said formed complex; and

detecting the presence or amount of the formed complex in said second region in an amount indicative of the andropausal condition.

According to still a further aspect of the present invention there is provided a device for assessing andropausal status in a male subject, the device comprising;

a housing;

a support disposed within said housing having a first region where is located labelled clinical marker binding compound capable of binding with a clinical marker for andropausal status that is present in a sample from a male subject and applied to said first region, and a second region adjoining said first region where is located an antibody that recognizes clinical marker bound to the labelled clinical marker binding compound.

According to still a further aspect of the present invention there is provided a diagnostic kit for assessing andropausal status in a male subject, the kit comprising

a support having a first region where is located a labelled clinical marker-binding compound;

a second region adjoining said first region where is located an antibody that recognizes said labelled clinical marker-binding compound; and

instruction for use.

In a specific embodiment, the kit is manufactured commercially for home use by a patient and may additionally include packaging, instructions for use, biological fluid sample collector and/or dropper.

Useful markers for detection in the method and kit of the present invention include but are not limited to FSH, LH, androgens, androgen precursors and androgen metabolites. Precursors to testosterone include pregnenolone, progesterone, 17-hydroxypregnenolone, 17-hydroxyprogesterone, dehydroepiandrosterone (DHEA) and delta-4-androstene-3,17-dione. Testosterone and dihydrotestosterone metabolites include the 17-ketosteroids (androsterone & etiocholanolone), polar metabolites in the form of diols, triols, and conjugates, as well as estradiol. It is understood by those of skill in the art that the method and kit of the invention may be designed to include more than one clinical marker as recited above or a combination of clinical markers as taught. Where more than one clinical marker is being analyzed a suitable number and type of antibodies are present in the second region of the support. Alternatively, where one or more clinical marker is being analyzed separate strips may be provided each of which include one marker and a corresponding recognizing antibody. In this manner, the kit of the present invention contains multiple test strips.

The preferred biological fluid for use in the present invention is urine, however, it is understood by those skilled in the art that other body fluids may also be used such as for example, saliva, semen, perspiration, mucous, blood and plasma. It is preferred that the biological fluid be one that is readily collectable by non-invasive methods.

### BRIEF DESCRIPTION OF THE DRAWINGS

An embodiment of the present invention is described with reference to the following drawings in which:

FIG. 1 illustrates a cassette (A) that may be used in one embodiment to conduct the method of the invention and the cassette in the sealed form (B) in which it is delivered for use; and

FIG. 2 illustrates the results window of a cassette of the type shown in FIG. 1 indicating a positive result (A) and a negative result (B).

The drawings are for illustrative purposes only and are not to be construed to limit the scope of the present invention as described herein.

### DETAILED DESCRIPTION OF THE INVENTION

The present invention is a novel method, device and test kit system for diagnosing and assessing the condition of andropause and/or reduced gonadal function in males in a rapid, reliable, simple and non-invasive manner. The condition of andropause is oftentimes not recognized by a male until symptoms become problematic. Diagnosis is then only done by a physician using an invasive blood test which results take days to obtain. Only then can the condition be treated as required. Therefore, the present invention allows for a patient to be able to conduct a diagnostic test at home or other suitable private venue in a comfortable manner whenever the patient desires. Such home testing leading to earlier treatment of the condition and associated symptoms.

The method of the present invention is a non-invasive diagnostic method that can readily be practised preferably by a patient to determine the level of a certain clinical marker (or mixtures thereof) of andropause in their system. The method involves applying a fluid biological sample from the male patient to a support having a first region where a labelled clinical marker-binding compound is present. On a second region of the support, adjacent to the first region, antibodies which recognize the conjugated clinical marker being measured are present. Should the clinical marker be present in the patient's sample it would bind to the labelled clinical marker binding compound resulting in a labelled clinical marker-binding compound complex also referred to as conjugate. The fluid is then allowed to flow through the support to the second region where the complex (or conjugate) will bind to the antibody. If a sufficient amount of labelled and complexed clinical marker is detected in a concentration indicative of andropause in the sample then a visual result will be readily visible to the eye. The second region may or may not contain a control area to indicate that the test is viably detecting free labelled clinical marker.

The biological sample may be selected from the group consisting of urine, blood, plasma, serum, saliva and mixtures thereof. Preferably, the sample is a urine sample excreted upon rising in the morning, herein referred to as “first morning urine”. Use of such a sample prevents the occurrence of false results since highly concentrated urine will yield a false positive result and an overly dilute urine will yield a false negative result. Both of these results are due to an inaccurate reflection of the actual amount of hormone(s) within the system.

Clinical markers indicative of andropause or a hypogonadal state and useful for detection in the present invention include testosterone; testosterone precursors such as pregnenolone, progesterone, 17-hydroxypregnenolone, 17-hydroxyprogesterone, dehydroepiandrosterone (DHEA) and delta-4-androstene-3,17-dione; testosterone and dihydrotestosterone metabolites such as the 17-ketosteroids androsterone and etiocholanolone, polar metabolites in the form of diols, triols, and conjugates, as well estradiol. Levels of all these marker compounds in the serum and other bodily fluids will vary in relation to testosterone (especially those precursors directly synthesized in the testicles and most closely related to testosterone).

In addition to the steroid sex hormones (and their precursors and metabolites), other potentially useful markers for use in the present invention are the hormones FSH (follicle stimulating hormone), LH (luteinizing hormone), and GnRH (gonadotropin-releasing hormone). GnRH is a decapeptide that is synthesized in the hypothalamus and transported to the anterior pituitary gland where it stimulates the release of FSH and LH. FSH and LH are polypeptide hormones that stimulate testosterone and sperm production in the testicles. Testosterone directly inhibits the secretion of GnRH, FSH, and LH. Hence in the presence of decreased levels of testosterone, the gonadal secretion of GnRH, FSH, and LH rises. FSH has a longer serum half life than LH, hence it's rise tends to be more dramatic that LH. For this reason elevated serum FSH levels has been one of the main biological markers for menopause in women, and it is also quite reliably elevated in male andropause. In adult men the secretion of GnRH, FSH, and LH is quite pulsatile. As a result the production of the gonadal sex hormones also fluctuate quite widely through out the day. In the case of hormones that undergo pulsatile secretion (such GnRH, FSH, LH and testosterone), a single serum value may not be representative of mean plasma levels. In such instances it is necessary either to measure and average levels in several samples drawn at random or to pool aliquots of three or more samples of plasma drawn at twenty to thirty minute intervals for a single determination. This averaging effect occurs naturally as urine produced by the kidneys is collected and mixed in the bladder prior to a patient voiding to providing a urine sample. Measurement of the urinary excretion rate of a hormone or a hormone metabolite that reflects plasma levels or secretory rates offers certain advantages over the measurement of isolated plasma levels; for example, urinary excretion reflects the average of the plasma levels over the time of collection. This principle means that a single urine test for GnRH, FSH, LH and testosterone (or its precursors or metabolites) is much more likely to accurately represent average serum levels than a single serum test.

The support to which the sample is applied may be any support suitable to support a first region comprising a labelled clinical marker binding compound and a resulting complex (with bound clinical marker which is also referred to as a conjugate) as well as a second region comprising antibody to the clinical marker such as for example, human FSH. An example of suitable support is a cellulose ester with nitrocellulose being especially preferred. It is understood that nitrocellulose refers to nitric acid esters of cellulose which may be nitrocellulose alone or a mixed ester of nitric acid and other acids, in particular, aliphatic carboxylic acids having from one to seven carbon atoms with acetic acid being preferred. Such solid supports which are formed from cellulose esterified with nitric acid alone or a mixture of nitric acid and another acid such as acetic acid are often referred to as nitrocellulose paper. Although nitrocellulose is a preferred material for the support, it is to be understood that other materials having a surface area sufficient for supporting a clinical marker-binding compound in a suitable concentration may also be employed for producing such solid supports including but not limited to nylon.

It is preferred that an absorbent pad be provided on the support in the first region in which the indicator-immune complex binder conjugate is found. Such absorbent pad acts to absorb the biological fluid sample such as urine thus allowing slow wetting of the underlying nitrocellulose support. The biological fluid sample then travels by capillary action along the nitrocellulose support to the second region. Thus an absorbent pad acts as a reservoir for the conjugate and to hold sufficient biological sample to wet the nitrocellulose with little if any flow over from the first region. A second absorbent pad may be provided downstream of the second region in order to absorb any excess biological sample and thus act as a waste reservoir. It is also acknowledged that the invention can be practised without the presence of an absorbent pad in the first region.

The support may be provided in protected form, for example, encased in an inert protective covering made of any suitable material type material such as plastic for example. In one embodiment, the support is provided in a cassette-like structure which houses the first and second regions, each of which is accessible only through a small window on one face of the cassette. In this embodiment, the sample can be applied to the window with minimal possibility of damage to the first and second regions. Provision of the medium protected in this way is particularly appropriate and desirable for direct patient use.

The support can also be provided in a partially-protected form provided on a substrate in a sheet form being in the form of a card, test strip or dip stick in which part of the support to which the biological fluid is applied, extends beyond the cassette. This form of the support is also suitable for direct patient use.

### EXAMPLES

The examples are described for the purposes of illustration and are not intended to limit the scope of the invention.

Methods of chemistry, protein and peptide biochemistry and immunology referred to but not explicitly described in this disclosure and examples are reported in the scientific literature and are well known to those skilled in the art.

### Example 1

### Performing the Test—Using FSH as Andropause Marker

To conduct the test, a urine sample is preferably applied to the first region of the support in an amount of about 1 to 2 mls in order to yield accurate results. The sample can be conveniently applied to the support using a dropper in which case 3-5 drops of sample are sufficient. The use of more than 1-2 mls (or 3-5 drops) would not have any adverse affect to the test, but would be undesirable when applied to a medium in a protective covering since excess sample would spill over the edges of the covering. Use of less than this amount of sample may fail to yield any result since there may be insufficient sample to wet the support thereby preventing a continuous flow of the sample to the second region from which the diagnostic result is obtained.

The urine sample is applied to the first region which has labelled FSH-binding compound. In general, the amount of binding compound present is greater than the amount of antigen present in the sample of urine. One skilled in the art would readily be able to determine how much binding compound is to be used in the first region. The FSH-binding compound may be labelled in any manner conventional in the art which allows identification of a reaction but which does not require the addition of any further compounds or reactants in order to conduct this identification. In this way, the method can be conducted in a single step and is particularly suited for direct use by a patient, but may also be used by a professional in a laboratory setting. In one example, calorimetric identification may be used. Colorimetric identifiers such as colloidal gold are useful for this type of identification. One skilled in the art would readily know the type of colorimetric identifiers that are suitable for use in the present invention.

The FSH-binding compound for binding the clinical indicator (forming the conjugate) may be any compound capable of binding to an immune complex formed by FSH and an antibody thereto (FSH immune complex). One example of a suitable FSH-binding compound is bacterial (staphylococcal) protein A. Protein A binds specifically to anti-FSH on formation of the FSH immune complex due to a conformational change in anti-FSH structure that occurs on formation of the FSH immune complex.

The FSH-binding compound may also be selected from any compound capable of binding to FSH which does not preclude or necessitate the binding of FSH to a further compound at a different site. Thus, the FSH-binding compound may be an antibody to either the beta or alpha subunit which does not prevent the unbound subunit from binding to a further compound. Preferably, the FSH-binding compound is an antibody to the beta-subunit of FSH. An example of such an FSH-binding compound is monoclonal antibody to the beta subunit of FSH, also referred to herein as monoclonal anti-beta FSH. In this case, monoclonal anti-beta FSH binds to the beta subunit of FSH leaving the alpha subunit of FSH free for further binding. Use of monoclonal anti-beta FSH advantageously provides specificity for the beta-subunit of FSH. This is important since it is the beta-subunit that confers on FSH its biological activity. In a most preferred embodiment, the monoclonal anti-beta FSH is a monoclonal mouse anti-human beta FSH.

The first region on the support retains the conjugate prior to the application of the urine sample such that the conjugate is readily adsorbed by urine on application of the urine sample. An absorbent pad is used in the first region in order to act as a reservoir for the conjugate and to hold sufficient urine to wet the underlying nitrocellulose support. The conjugate is bound onto the pad by surface tension and is readily picked up by the applied urine sample. The adsorbent pad can be made of any suitable material as is understood by one skilled in the art so long as it can retain the conjugate and be mounted to a first region of the support.

The second region on the support is located adjacent to the first region. This region contains immobilized antibody to FSH, or anti-FSH, that is capable of binding FSH alone, or capable of binding either the alpha or beta subunit of FSH. In general, excess antibody is used and present in the second region and suitable quantities can be determined by one skilled in the art. In a preferred embodiment, the second region contains immobilized antibody to the alpha subunit of FSH. An example of an antibody that is appropriate for immobilization at the second region is polyclonal antibody to the alpha subunit of FSH, also referred to herein as polyclonal anti-alpha FSH. Use of an antibody to alpha subunit of FSH is advantageous in that the alpha subunit provides numerous binding sites for the antibody, thereby permitting the binding of more than one antibody. Use of polyclonal antibody is also advantageous in that it is less specific and can bind at the numerous sites available on the alpha subunit. Both of these features increases the sensitivity of the present method. In a most preferred embodiment, the polyclonal anti-alpha FSH is a polygonal goat anti-human alpha FSH.

Anti-FSH is coated on the second region in an amount sufficient to detect FSH at a concentration indicative of andropause. This concentration will vary with the individual. Preferably, a FSH concentration of about at least 6-12 IU/L (International Units per litre of sample) is considered to be indicative of andropause. The amount of anti-FSH required to detect this concentration of FSH can readily be determined using dose-response analyses conventionally used by those of skill in the art. Detection limits for other useful clinical indicators can also be similarly determined.

In practice, the sample is applied to the first region and picks up the binding compound to form a conjugate as it moves by capillary action to the second region. If there is FSH in the sample, then it will bind with the anti-FSH bound on the second region to form an immune complex. Depending on the FSH-binding compound used to form the conjugate, the conjugate will either couple to the anti-FSH/FSH immune complex formed on the second region via the FSH-binding compound, or will have already coupled to the FSH prior to its binding with the anti-FSH secured to the second region. Either way, the labelled conjugate is retained in the second region thereby permitting detection of the label, and thus, detection of FSH in the sample. If no FSH is present in the sample, there will be no binding of the FSH-binding compound and the labelled conjugate will not be retained in the second region. If there is less than the cut off level of FSH in the sample then there will be binding of the FSH-binding compound and retention of the labelled conjugate but this will be insufficient to detect and thus a negative result.

### Example 2

### Commercial Kit

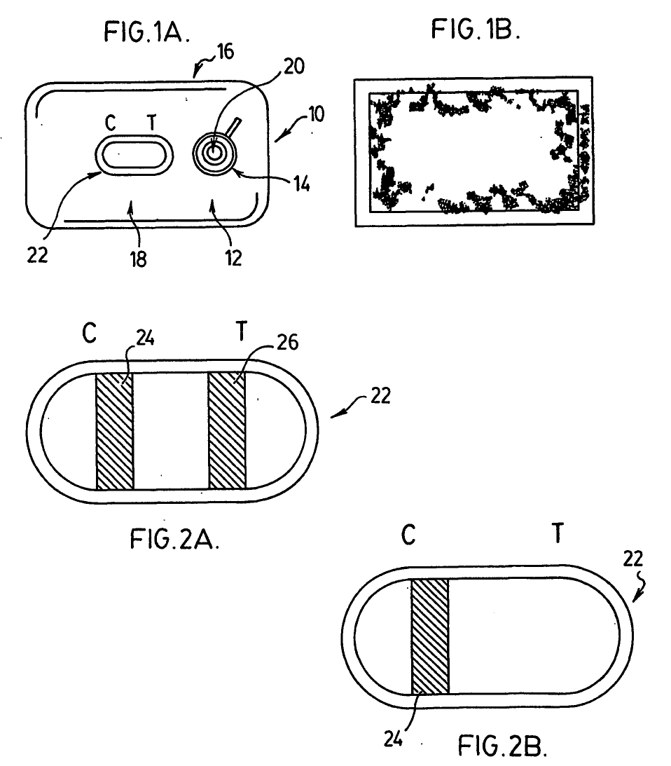

In one embodiment of the invention, the method is conducted using a device (referred to herein as a cassette) 10 as shown in FIG. 1A. The cassette is made of any standard cassette-making material, for example, plastic. The cassette houses a first region 12 having an absorbent pad 14 to which is present dried colloidal gold-monoclonal anti-beta FSH conjugate. This first region 12 is mounted on a nitrocellulose membrane 16. The nitrocellulose membrane 16 extends to form a second region 18 located adjacent to the absorbent pad 14 and coated with polyclonal anti-alpha FSH. The cassette has two windows, the first of which is termed a “drop zone window” 20 and is located at the first region 12. This is where the sample is applied. The second window is termed the “results window” 22 and it is located at the second region 18. In the results window 22 there is a control area 24 labelled “C” and a test area labelled “T” 26. The control area 24 on the nitrocellulose is impregnated with polyclonal anti-immunoglobulin. Accordingly, free labeled anti-beta FSH which is always present in excess will bind to the anti-immunoglobulin at the control area producing a coloured control line regardless of whether there is FSH in the applied sample or not. This will occur because in the application of samples not containing FSH, the labelled anti-beta FSH will remain free. In the application of samples containing FSH, there will also be free anti-beta FSH because excess is applied to the first region in order to ensure the presence of a control. Although, the use of anti-immunoglobulin G is preferred, anti-immunoglobulin A, anti-immunoglobulin M, anti-immunoglobulin D and anti-immunoglobulin E may also be used at the control line. The test area 26 (in the second region) is coated with only polyclonal anti-alpha FSH and will only produce a visible coloured line if FSH of at least 6-12 IU/L is present in the sample.

As will be appreciated by those of skill in the art, the present method is semi-quantitative in nature such that a positive result (FIG. 2A) is obtained at a specific andropause-indicating concentration of FSH, such as at least 6-12 IU/L. FSH concentrations below this indicative concentration will register a negative result (FIG. 2B).

Multiple test areas of increasing anti-FSH concentration could be incorporated into the second region in order to make the method more quantitative, i.e. in order to more readily identify the exact concentration of FSH in a patient's biological sample, rather than simply an indication that FSH concentration is at least 6-12 IU/L.

In another aspect of the present invention, a kit is provided that is useful to diagnose andropausal status. The kit comprises a medium having a first region comprising a conjugate. The conjugate comprises a labelled FSH-binding compound, and a second region comprising antibody to human FSH. The first and second regions of the medium are adjacent so as to allow continuous flow of the sample from the first to the second region.

In one embodiment of the invention, as described in detail above, the kit comprises a cassette-like structure (see FIG. 1A) which houses the first and second regions. To maintain the cassette during storage and prior to its use, it is preferably sealed in a foil wrapping as shown in FIG. 1B. The nature of the first and second regions is as already described supra.

For the convenience of the user, the kit may additionally include instructions, a container for collecting the urine sample and means to apply an appropriate amount of the sample to the application site or “drop zone” on the cassette such as a dropper. The kit may be suitably packaged and labelled as desired. The kit is disposable after use.

As will be appreciated by those of skill in the art, the invention may be varied in any number of ways as would be apparent to a person skilled in the art and all obvious equivalents and the like are meant to fall within the scope of this description and claims. The description is meant to serve as a guide to interpret the claims and not to limit them unnecessarily,

We claim:

1. A non-invasive method for the diagnosis of andropause in a male subject, the method comprising:
conducting a biological assay on a male fluid sample and determining the presence of one or more clinical markers of andropause at a concentration indicative of andropause.

2. The method of claim 1, wherein said biological assay is an immunological assay.

3. The method of claim 2, wherein said immunological assay comprises an antibody specific for binding to a labelled clinical marker binding compound conjugate.

4. The method of claim 3, wherein said clinical marker binding compound is a labelled FSH—binding compound which upon FSH binding forms a labelled conjugate.

5. The method of claim 4, wherein said clinical marker binding compound is selected from the group consisting of Protein A, antibody to alpha subunit of FSH and antibody to beta subunit of FSH.

6. The method of claim 5, wherein said labelled clinical marker binding compound conjugate is visually detectable at a level indicative of andropause.

7. The method of anyone of claims 1-6, wherein said fluid sample is selected from the group consisting of urine, saliva, semen, perspiration, mucous, blood, plasma and serum.

8. The method of claim 7, wherein said fluid sample is urine.

9. The method of claim 8, wherein said clinical marker is selected from the group consisting of FSH, LH, androgens, androgen precursors and androgen metabolites.

10. The method of claim 9, wherein androgen precursors are selected from the group consisting of pregnenolone, progesterone, 17-hydroxypregnenolone, 17-hydroxyprogesterone, dehydroepiandrosterone (DHEA) and delta-4-androstene-3,17-dione.

11. The method of claim 9, wherein androgen metabolites are selected from the group consisting of testosterone metabolites, dihydrotestosterone metabolites, polar metabolites, conjugates and estradiol.

12. A diagnostic method for determining the andropausal status of a male subject by non-invasive detection of a clinical marker of andropause, the method comprising the steps of:
applying a biological fluid sample of said subject to a first region on a support where is located a labelled clinical marker-binding compound, wherein any clinical marker present in said biological fluid sample binds to said labelled clinical marker-binding compound forming a conjugate;
allowing the biological fluid sample to flow to a second region adjacent said first region where is located an antibody that recognizes said formed conjugate; and
visually detecting the presence or amount of the formed conjugate in said second region in an amount indicative of the andropausal condition.

13. The method of claim 12, wherein said clinical marker is selected from the group consisting of FSH, LH, androgens, androgen precursors and androgen metabolites.

14. The method of claim 13, wherein androgen precursors are selected from the group consisting of pregnenolone, progesterone, 17-hydroxypregnenolone, 17-hydroxyprogesterone, dehydroepiandrosterone (DHEA) and delta-4-androstene-3,17-dione.

15. The method of claim 13, wherein androgen metabolites are selected from the group consisting of testosterone metabolites, dihydrotestosterone metabolites, polar metabolites, conjugates and estradiol.

16. The method of claim 13, wherein said marker is FSH.

17. The method of claim 16, wherein said fluid sample is selected from the group consisting of urine, saliva, semen, perspiration, mucous, blood, plasma and serum.

18. The method of claim 17, wherein said fluid sample is urine.

19. A method for detecting the presence of an andropause clinical marker as a predictive indicator of andropause in a male patient, the method comprising;
conducting a biological assay for detecting the presence of an andropause clinical marker in a biological fluid sample from said patient; and
upon detecting the andropause clinical marker in said sample classifying said sample as positive which indicates an andropausal condition in said patient.

20. The method of claim 19, wherein said biological assay is an immunological assay.

21. The method of claim 20, wherein said immunological assay comprises an antibody specific for binding to a labelled clinical marker binding compound conjugate.

22. The method of claim 21, wherein said clinical marker binding compound is a labelled FSH—binding compound which upon FSH binding forms a labelled conjugate.

23. The method of claim 22, wherein said clinical marker binding compound is selected from the group consisting of Protein A, antibody to alpha subunit of FSH and antibody to beta subunit of FSH.

24. The method of claim 23, wherein said labelled clinical marker binding compound conjugate is visually detectable at a level indicative of andropause.

25. The method of claim 24, wherein said fluid sample is selected from the group consisting of urine, saliva, semen, perspiration, mucous, blood, plasma and serum.

26. The method of claim 25, wherein said fluid sample is urine.

27. The method of claim 26, wherein said clinical marker is selected from the group consisting of FSH, LH, androgens, androgen precursors and androgen metabolites.

28. The method of claim 27, wherein androgen precursors are selected from the group consisting of pregnenolone, progesterone, 17-hydroxypregnenolone, 17-hydroxyprogesterone, dehydroepiandrosterone (DHEA) and delta-4-androstene-3,17-dione.

29. The method of claim 27, wherein androgen metabolites are selected from the group consisting of testosterone metabolites, dihydrotestosterone metabolites, polar metabolites, conjugates and estradiol.

30. A device for assessing andropausal status in a patient, the device comprising;
a housing;
a support disposed within said housing having a first region where is located labelled clinical marker binding compound capable of binding with a clinical marker for andropausal status that is present in a sample from a male subject and applied to said first region, and a second region adjoining said first region where is located an antibody that recognizes clinical marker bound to the labelled clinical marker binding compound.

31. The device of claim 30, wherein said housing has a first and second aperture for exposing said first and a second aperture for exposing said second region.

32. The device of claim 31, wherein said housing is made of a plastic material.

33. The device of claim 32, wherein said device additionally comprises an absorbent pad overlying the first region of said support.

34. The device of claim 33, wherein said device additionally comprises an absorbent pad overlying said second region of said support.

35. The device of claims 33 or 34 wherein said absorbent pad is a cellulose ester.

36. The device of claim 35, wherein said cellulose ester is nitrocellulose.

37. The device of claim 30, wherein said labelled clinical marker binding compound is colloidal gold-monoclonal anti-beta FSH conjugate.

38. The device of claim 37, wherein said antibody is polyclonal anti-alpha FSH.

39. The device of claim 32, wherein said device additionally comprises a control region in said second region of said support where is located polyclonal anti-immunoglobulin.

40. A diagnostic kit for assessing andropausal status in a patient, the kit comprising
a support having a first region where is located a labelled clinical marker-binding compound;
a second region adjoining said first region where is located an antibody that recognizes said labelled clinical marker-binding compound;
instructions for use.

41. The kit of claim 40, wherein said labelled clinical marker binding compound is colloidal gold-monoclonal anti-beta FSH conjugate.

42. The kit of claim 41, wherein said antibody is polyclonal anti-alpha FSH.

43. The kit of claim 42, wherein said device additionally comprises a control region in said second region of said support where is located polyclonal anti-immunoglobulin.

44. The kit of claim 43, wherein said kit is provided within packaging,

45. The kit of claim 44, further comprising a sample dropper and/or sample collector.
